(19) United States
(12) Patent Application Publication
(10) Pub. No.: US 2007/0233012 A1
(43) Pub. Date: Oct. 4, 2007

(54) Multi-chamber spray container and system

(76) Inventors: Andrew J. Lerrick, Highland Park, IL (US); Olivia R. Lerrick, Highland Park, IL (US)

Correspondence Address: SEYFARTH SHAW LLP, 131 S. DEARBORN ST., SUITE2400, CHICAGO, IL, 60603-5803, US

(21) Appl. No.: 11/710,891

(22) Filed: Feb. 26, 2007

Related U.S. Application Data
(60) Provisional application No. 60/776,716, filed on Feb. 24, 2006.

(51) Int. Cl. B67D 5/56; B67D 5/52; A61M 31/00; A61M 5/00; A61M 37/00

(52) U.S. Cl. 604/191; 604/94.01

(57) ABSTRACT

A disposable or reusable spray container including a housing; at least two storage chambers in the housing, each of the storage chambers containing one or more different ingredients and an actuator on the housing, wherein activation of the actuator causes simultaneous emission of the ingredients from the storage chambers of the container.

### PRIORITY CLAIM

This application claims priority to and the benefit of U.S. Provisional Application Ser. No. 60/776,716, filed Feb. 24, 2006, the entire contents of which are incorporated herein.

### BACKGROUND

Spray containers come in different shapes and sizes. There are also many different sprays used for many different purposes. Some sprays use a chemical such as an aerosol to help propel the contents of the spray container out of the container upon pressing a button or other actuator. Many household cleaners and deodorants are manufactured in the form of aerosol-based sprays. Other types of spray containers utilize a change in pressure to emit the contents of the container. For example, many medications are administered as sprays using a pressure-based system to avoid the use of aerosols and other ingredients that might be dangerous if inhaled, ingested or applied topically and can affect the potency or stability of the medication being administered. These sprays are emitted from their containers when the pressure in the container exceeds a designated level. Many times the user actuates or pumps a pumping device on a spray container one or more times to create the necessary pressure within the container to emit the contents from the container. Many nasal sprays can be administered using a pressure-based system, such as FlonaseR (fluticasone), Rhinocort® (budesonide), Nasacort® (triamcinolone), Nasonexe® (mometasone), Nasarel® (flunisolide), Beconase® (beclomethasone). These and other nasal sprays are administered directly into one or both of the nasal cavities to help alleviate the symptoms commonly associated with allergies, colds, flu, and the like.

Many of the nasal sprays have an unpleasant or immediate aftertaste because of the medicine contained in the spray. Unlike many pediatric oral preparations, which come in solution or suspension formulations containing ingredients that mask the flavor of the medication, such as Tylenol® and Augmentin®, spray preparations often contain a single medication, with or without a preservative, but lack a flavor enhancer for reasons of chemical incompatibility or instability. This can be especially problematic with children who may not want to use the spray because of the unpleasant taste.

Therein lies the problem: spray formulations typically contain only one medication per container For example, a person may have one spray container containing one medicine and another different spray container containing another different medicine. The person, therefore, has to spray each container separately into their nose, which can be time consuming, burdensome, and lead to non-compliance in the use of the medication.

Furthermore, having more containers means that a person has to keep track of those containers and is more likely to misplace or lose those containers.

Accordingly, there is a need for an improved spray container which overcomes the above problems.

### SUMMARY

The present invention is directed to a disposable or reusable spray container including at least two storage chambers and an actuator, wherein activation of the actuator causes the simultaneous emission of the ingredients contained in the storage chambers.

In an embodiment, at least one of the ingredients is a liquid medication.

In an embodiment, the liquid medication is at least one of: Azelastine and a steroid.

In an embodiment, at least one of the fluids is a flavor-enhanced fluid.

In an embodiment, at least one of the ingredients is at least one of the following: a powder, a perfume, a glitter, a paint, and a breath spray.

In an embodiment, at least one of the chambers is angled.

In an embodiment, the housing has a conical shape.

In an embodiment, the actuator includes finger depressions.

In an embodiment, at least one of the chambers contains two medications.

In an embodiment, at least one of the chambers contains one medication and a flavor-enhanced fluid.

In an embodiment, at least one of the chambers contains two medications and a flavor-enhanced fluid.

Another embodiment provides a disposable or reusable nasal spray container including a housing defining a plurality of openings and a plurality of chambers in the housing, where each of the chambers is in communication with one of the openings and at least two of the chambers store at least one different ingredient. The spray container includes an actuator on the housing wherein activation of the actuator causes simultaneous propulsion of the ingredients from the chambers out of the openings and into a nasal cavity of a person.

In an embodiment, at least one of the ingredients is a liquid medication.

In an embodiment, the liquid medication is at least one of: Azelastine and a steroid.

In an embodiment, at least one of the ingredients is a flavor-enhanced fluid.

In an embodiment, at least one of the ingredients is at least one of the following: a powder, a perfume, a glitter, a paint, and a breath spray.

In an embodiment, at least one of the chambers is angled.

In an embodiment, the housing has a conical shape.

In an embodiment, the actuator includes a flange.

In an embodiment, the flange includes at least one finger depression.

A further embodiment provides a method of spraying multiple ingredients from a container including providing a container including at least two storage chambers and an actuator, each of the storage chambers containing a different ingredient and activating the actuator to simultaneously propel the ingredients from the storage chambers and out of the container.

Accordingly, an advantage of the present invention is to provide a multi-chamber spray container that simultaneously emits multiple sprays in a single activation of the container.

Another advantage is to provide spray container having multiple medicinal sprays and/or flavor enhancers to enable a patient to spray multiple medications and/or flavor enhancers in a single activation of the container.

A further advantage is to provide a multi-chamber spray container where at least one chamber includes medicine and at least one chamber includes a flavor-enhanced fluid.

Other objects, features and advantages of the invention will be apparent from the following detailed disclosure, taken in conjunction with the accompanying sheets of drawings, wherein like numerals refer to like parts, elements, components, steps and processes.

### BRIEF DESCRIPTION OF THE DRAWINGS

FIG. 1 is a perspective view of a multi-chamber spray container of the present invention where the spray container includes three chambers, and where the internal components of the container are shown.

FIG. 2 is fragmentary perspective view of the embodiment of FIG. 1.

FIG. 3 is a side view of the embodiment of FIG. 1.

FIG. 4 is a top view of the embodiment of FIG. 1.

FIG. 5 includes perspective views of alternative embodiments of the container of FIG. 1 where the internal components of the container are shown.

FIG. 6 is a perspective view of an additional alternative embodiment of the container of FIG. 1 where the container includes a different exterior design and where the internal components of the container are shown.

FIG. 7 is a perspective view of another embodiment of the multi-chamber spray container of the present invention where the container includes two chambers and where the internal components of the container are shown.

FIG. 8 is fragmentary perspective view of the embodiment of FIG. 7.

FIG. 9 is a top view of the embodiment of FIG. 7.

### DETAILED DESCRIPTION

The present invention is directed to a spray container and more specifically to a multi-chamber spray container which stores different ingredients and which simultaneously emits the ingredients from the container.

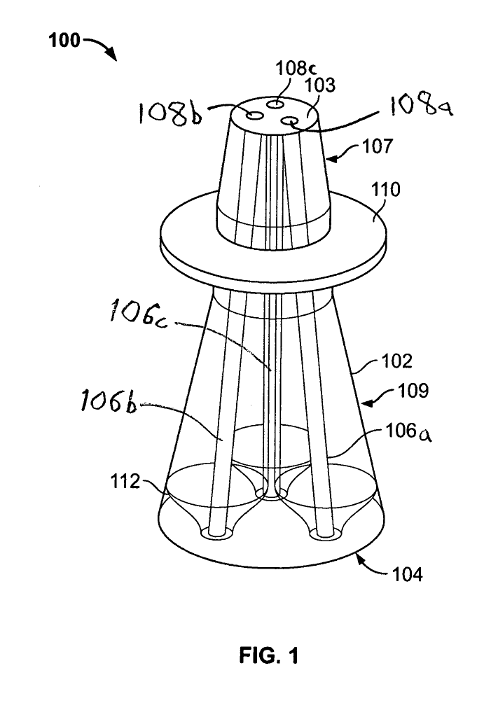

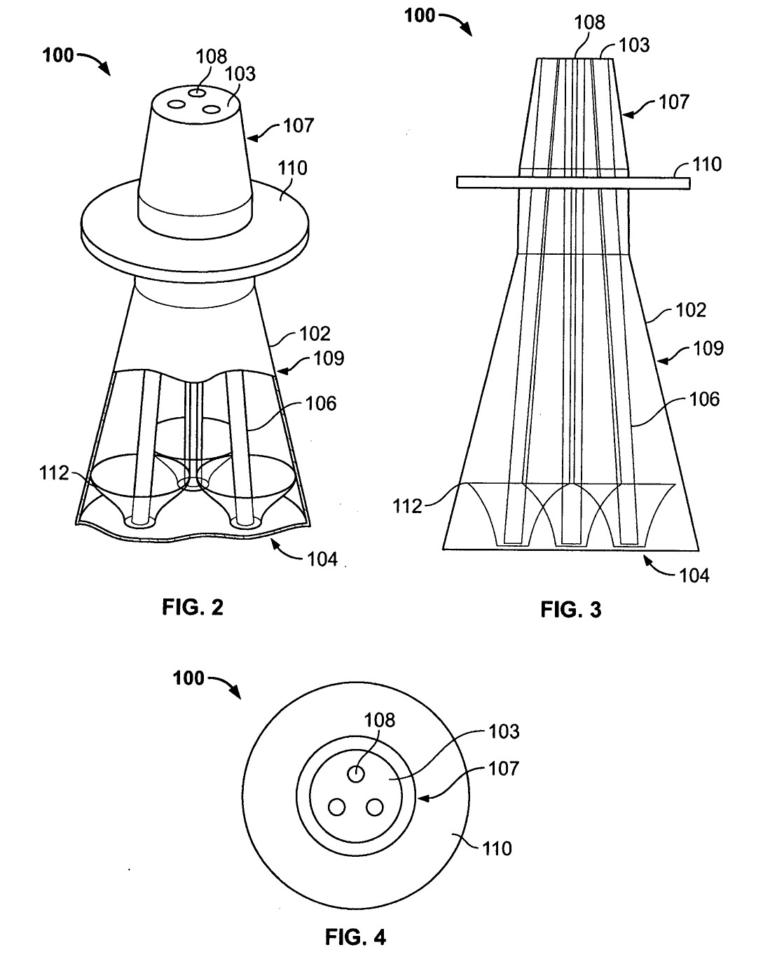

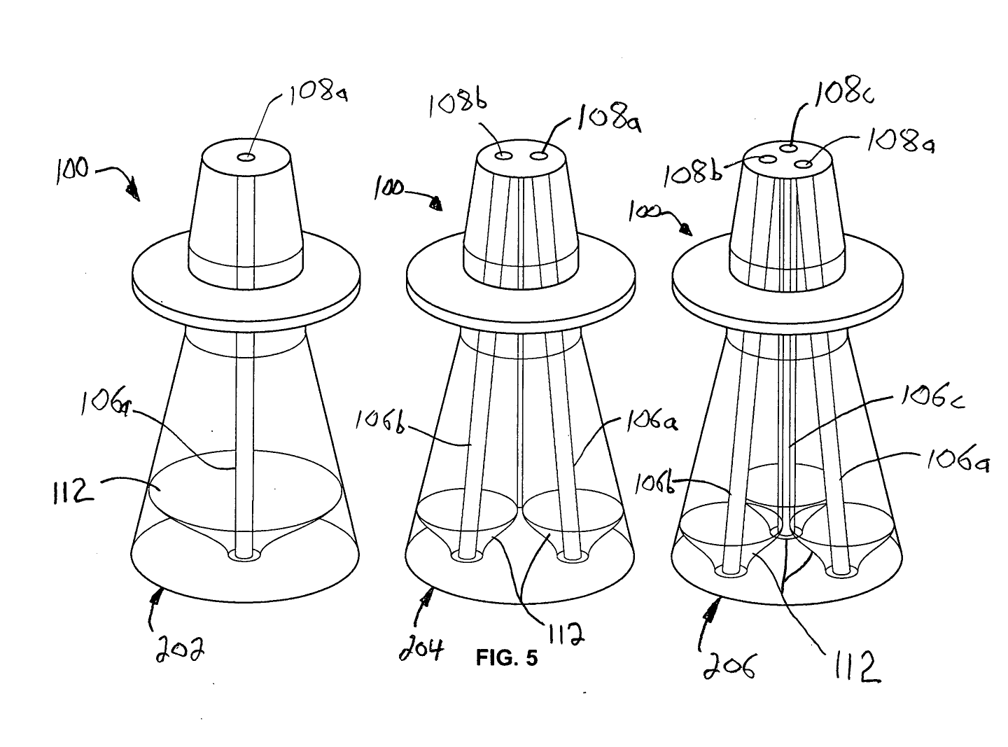

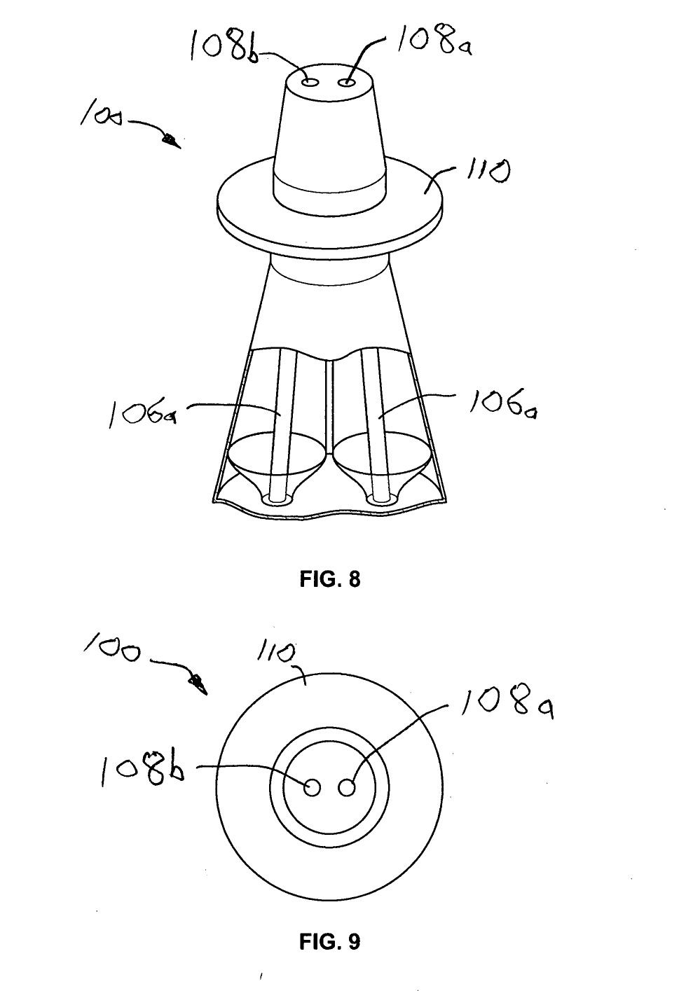

Referring to FIGS. 1-9, multiple embodiments of spray containers are shown where the container 100 includes a body or housing 102 which is generally cone-shaped and has a flat bottom surface 104 so that the container can stand upright on a underlying surface such as a tabletop or counter. It should be appreciated that the housing 102 may be any suitable size or shape. At least two cylindrical chambers 106a,b,c are carried in the housing 102 (except for the historical rendering of container 202 in FIG. 5). It should be appreciated that the chambers 106a,b,c may be any size or shape. FIG. 1 shows a three-chamber spray container and FIG. 7 shows a two chamber container. Each cylindrical chamber 106a,b,c stores a different substance, ingredient or fluid. In an embodiment, each chamber 106a,b,c includes a different medicine. Each of the chambers 106a,b,c are positioned inside the housing 102 and extend at an angle upwardly to the top surface 103 of the housing. One end of each of the chambers 106a,b,c is in communication with one of the openings 108a,b,c of the top surface 103. A generally cylindrical flange 110 is attached to the upper end 107 of the housing to form an actuator. The upper end 107 is moveable with respect to the lower end 109. Therefore, when a user presses downwardly on the upper end (i.e., the actuator) 107, this creates pressure within the housing so that the fluids stored in the chambers 106a,b,c are emitted from the chambers. More specifically, upon actuation, one or more of the ingredients or fluids are simultaneously propelled from the chambers 106a,b,c and outside of the container 100.

In the above embodiments, a medicine or other substance is contained or stored in the chambers 106a,b,c. For example, one or more of the chambers 106a,b,c may include at least one of a steroid, Astelin® (Azelastine), Flonase®, Rhinocort®, Nasacort®, or Nasonex®. Other fluids, substances or ingredients may also be contained in a chamber such as powders, perfume, glitter, paint, breath spray and other suitable propellants.

As stated above, FIG. 1 depicts a three chamber 106a,b,c, three hole 108a,b,c bottle or container. Similar views can be extrapolated for single- and double-chamber, single- and double-hole bottles in the same orientation. The wide, round base provides optimal balance. The spray container 100, in an embodiment, is intended for use as a nasal spray. Therefore, the narrow top of the container is designed to fit into the nose of a person. Alternatively, the top of the container can be sized and shaped to fit into other openings depending on the use and purpose of the container.

The vertical neck of the body 102 provides room for the plunger or actuator 107 to glide up and down. The “Elizabethan” collar or flange 110 can be modified in two ways: (1) one version looks more like a “pogo stick” with horizontal extensions, on which the middle and pointer fingers would rest, or (2) another version includes curved extensions, in which fingers fit into the “depression”. All three shapes allow the fingers to depress the plunger 107, causing the pressure to increase inside the chambers, propelling the fluids from the chambers.

The chambers 106a,b,c can be: “tilted” so that they slope at an angle parallel to the external periphery of the chamber. The current bottle design can be modified to have both the “pogo stick” plunger versions (straight and unshaped) and the tilted chambers with angled funnels 112.

FIG. 5 depicts the single 202, double 204, and triple chamber 206 “standard” models. Two modifications are possible, among others, including two “pogo stick” finger depressor styles (straight or u-shaped) as well as the “straight chambers” and “tilted chambers with angled funnels.”

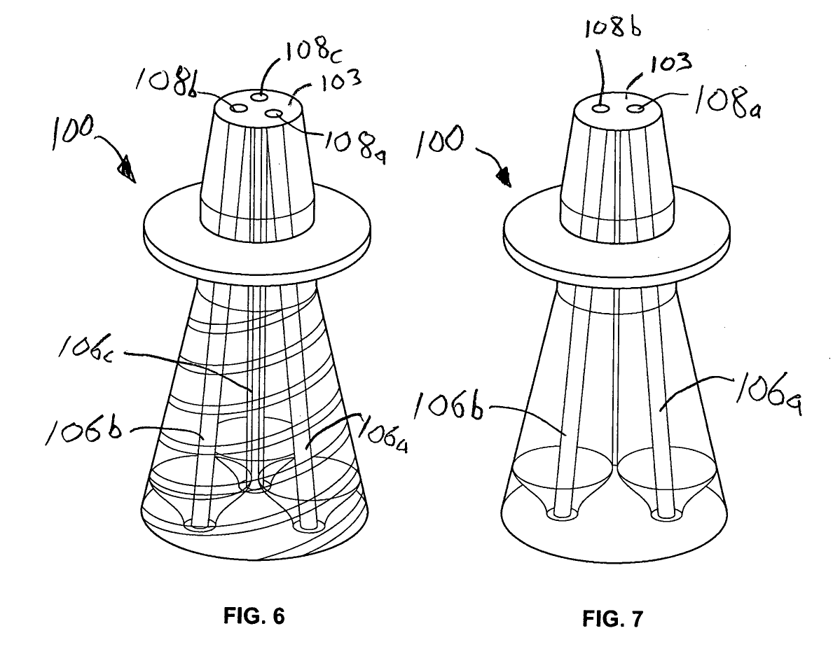

FIG. 6 shows the three chamber container or system of FIG. 1 having a different aesthetic design such as stripes or striping. The striping on different containers or bottles is intended to be of a different color or design, depicting different ingredients contained in the containers. Again, the colors and stripes are for marketing purposes to improve the appearance of the spray containers. It should be appreciated that any suitable design or designs such as other stripes, colors, textures, or other indicia, may be used on the container to enhance the appearance of the container or to indicate the ingredient or ingredients contained in a container.

The purpose of the “single chamber system” such as the spray container 202 of FIG. 5 is predominantly historical and introduces the internal design of the bottle. The exterior shape is generally cone-shaped with a narrower top end, and the internal funnel 112 allows the spray bottle to draw into the tube “every last drop” of the chambers content.

As stated above, the invention includes two- and three-chamber systems such as those shown in FIGS. 1 and 7, which permits simultaneous propulsion of the chambers' contents through the nozzle for concurrent or simultaneous emission and administration of the propelled ingredient. In this instance, a liquid medication, such as Azelastine and/or a steroid with or without a flavor-enhanced fluid or flavor enhancer can be stored in the chambers. Additionally, powders, perfume, glitter, paint, breath spray, etc. can all be propellants. These other agents are intended to be propelled to body sites deemed safe and appropriate with respect to internal or external use. In an embodiment, at least one of the chambers contains two medications. In another embodiment, at least one of the chambers contains one medication and a flavor-enhanced fluid. In a further embodiment, at least one of the chambers contains two medications and a flavor-enhanced fluid.

The angled chamber allows the internal chambers to have a larger volume. Moreover, the angle of the chambers vary based on whether the chambers reach the bottom of the funnels 112.

The circular finger flange 110 is an aesthetically appealing design as well as being functional. The “pogo stick” design alternatives can either be straight across or have a single depression on each side of the flange to allow the pointer finger and middle finger of a person's hand to be placed on the flange on each side of the central nozzle (the thumb is placed underneath the bottom of the hand held bottle or container 100).

The invention pertains to a spray container system having two or more chambers. The mechanism by which the fluid is propelled is not limited to that shown in the figures (i.e., a squeezable bulb). The chambers are configured to provide an external conical shape for stability and easy grip-ability. Tubes (not shown) leading from each chamber direct a flow of fluid to a delivery tip where the fluid is discharged to the nasal cavity. The delivery tip of each tube is positioned to discharge fluid out of the openings 108a,b,c to allow mixing of the fluids externally (i.e., within the nasal cavity). Any dispensing method may be used, such as a pump, aerosol spray or the like.

It should be appreciated that the container described above can be used as a nasal spray or for other purposes such as an ear spray, an oral spray, a hair spray, or any other suitable spray.

The embodiments set forth in the foregoing description and accompanying drawings are offered by way of illustration only and not as a limitation. While particular embodiments have been shown and described, it will be apparent to those skilled in the art that changes and modifications may be made without departing from the broader aspects of the applicants' contribution. The actual scope of the protection sought is intended to be defined in the following claims when viewed in their proper perspective based on the prior art.

The invention is claimed as follows:

1. A disposable or reusable spray container comprising:
a housing;
at least two storage chambers in the housing, each of the storage chambers containing a different ingredient; and
an actuator on the housing, wherein activation of the actuator causes simultaneous emission of the ingredients from the storage chambers.

2. The spray container of claim 1, wherein at least one of the ingredients is a liquid medication.

3. The spray container of claim 2, wherein the liquid medication is at least one of: Azelastine and a steroid.

4. The spray container of claim 1, wherein at least one of the ingredients is a flavor enhanced fluid.

5. The spray container of claim 1, wherein at least one of the ingredients is at least one of the following: a powder, a perfume, glitter, paint, and breath spray.

6. The spray container of claim 1, wherein at least one of the chambers is angled.

7. The spray container of claim 1, wherein the housing has a conical shape.

8. The spray container of claim 1, wherein the actuator includes at least one finger depression.

9. The spray container of claim 1, wherein at least one of the chambers contains two medications.

10. The spray container of claim 1, wherein at least one of the chambers contains one medication and a flavor-enhanced fluid.

11. The spray container of claim 1, wherein at least one of the chambers contains two medications and a flavor-enhanced fluid.

12. A disposable or reusable nasal spray container comprising:
a housing defining a plurality of openings;
a plurality of chambers in the housing, each of said chambers in communication with one of the openings, at least two of the chambers containing at least one different ingredient; and
an actuator on the housing wherein activation of the actuator causes simultaneous propulsion of at least one of the ingredients contained within the chambers out of the openings and into a nasal cavity of a person.

13. The spray container of claim 12, wherein at least one of the ingredients is a liquid medication.

14. The spray container of claim 13, wherein the liquid medication is at least one of: Azelastine and a steroid.

15. The spray container of claim 12, wherein at least one of the ingredients is a flavor-enhanced fluid.

16. The spray container of claim 12, wherein at least one of the chambers is angled.

17. The spray container of claim 12, wherein the actuator includes a flange.

18. The spray container of claim 17, wherein the flange includes at least one finger depression.

19. A method of spraying multiple fluids from a container comprising:
providing a container including at least two storage chambers and an actuator, each of the storage chambers containing a different ingredient; and
activating the actuator to simultaneously propel the ingredients from the storage chambers and out of the container.
